(19) United States
(12) Patent Application Publication
(10) Pub. No.: US 2023/0149201 A1
(43) Pub. Date: May 18, 2023

(54) CRANIAL REMOLDING ORTHOSIS DEVICE, SYSTEMS, AND METHODS THEREOF

(71) Applicant: ORTHOMERICA PRODUCTS, INC., Orlando, FL (US)

(72) Inventors: Kourtney F. Smith, Eustic, FL (US); Christina Magaret Hinton, Altamonte Springs, FL (US); Robert Ernest Tetro, Lake Mary, FL (US); Andres Pazmino, Midlothian, VA (US)

(21) Appl. No.: 17/985,789

(22) Filed: Nov. 11, 2022

Related U.S. Application Data
(60) Provisional application No. 63/278,893, filed on Nov. 12, 2021.

(51) Int. Cl. A61F 5/058

(52) U.S. Cl. CPC A61F 5/05891

(57) ABSTRACT

A cranial deformation orthosis device for a head of an infant, including a rigid outer shell with an anterior portion detachably connected to a posterior portion. The anterior portion is shaped and configured to be donned on a forward portion of the head of the infant. The posterior portion is shaped and configured to be donned on an aft portion of the head of the infant. Each of the anterior portion and the posterior portion can include an inner surface having at least one lined zone having selectively positioned padding to prevent head growth and at least one unlined zone having a plurality of ventilation holes extended through the inner surface to an outer surface and selectively configured to allow growth or movement of the head to reshape the head of the infant.

### CROSS-REFERENCE TO RELATED APPLICATIONS

This application claims the benefit and priority to U.S. provisional patent application no. 63/278,893, filed Nov. 12, 2021, the contents of which are incorporated herein by reference in their entirety as if set forth verbatim.

### FIELD

This disclosure relates to an orthosis, particularly a cranial remolding orthosis device for remolding the head shape of young humans, including but not limited to infants and children (e.g., aged approximately 3 to 18 months).

### BACKGROUND

The human skull during early infancy is formed of bone plates interconnected by ligaments known as sutures. As a baby matures, the plates will fuse to form the final, permanent skull shape. Yet, before forming the permanent skull shape, the skull can be pliable or otherwise soft enough to be deformed by external pressure. For example, if the child's head is in one position resting against a firm surface for an extended period of time, the child can develop one or more flat regions or zones on their skull.

In certain respects, this process is understood as infant flat head syndrome. It is understood that infant flat head syndrome can present in a variety of ways, including but not limited to one or a combination of Deformational Plagiocephaly, Brachycephaly, and Scaphocephaly (also known as Dolichocephaly). In some instances, a baby can be fitted with a skull shaping helmet so as to promote skull shaping back to a typical or otherwise acceptable head shape. Usually these helmets include rigid shells with a foam interior liner which acts as a mold to promote a specific shape for the baby's skull to grow into.

Although previous helmets have been somewhat successful, prior approaches have certain disadvantages. For example, manufacturing prior helmets has been labor intensive and misused corresponding helmet materials. Prior approaches have also required numerous helmet alterations to promote gradual skull reformation, which in turn has also unnecessarily increased costs and helmet efficacy.

The solution of this disclosure resolves these and other drawbacks that will be apparent.

### SUMMARY

In some aspects, a cranial deformation orthosis device is disclosed. The device can include a rigid outer shell including an anterior portion detachably connected to a posterior portion, the anterior portion being shaped and configured to be donned on a forward portion of the skull of the infant and the posterior portion being shaped and configured to be donned on an aft portion of the skull of the infant. Each of the anterior portion and the posterior portion can include an inner surface having at least one lined zone including selectively positioned padding to prevent skull growth in a region of the skull aligned thereunder and at least one unlined zone configured to permit skull growth in a region of the skull aligned thereunder, the at least one unlined zone having a plurality of ventilation holes extended through the inner surface to an outer surface. In this respect, the at least one unlined zone can be configured to allow for space over areas where cranial growth is desired.

In some aspects, each of the anterior portion and the posterior portion contain a hollow void between inner and outer surfaces.

In some aspects, each size and shape of the at least one lined zone is assigned based on data of an exact three-dimensional scan of the skull of the infant, wherein the selectively positioned padding is configured to prevent further growth of the skull in a corresponding skull region when the device is worn.

In some aspects, each size and shape of the at least one asymmetric unlined zone is assigned based on data of an exact three-dimensional scan of the skull of the infant.

In some aspects, the ventilation holes of the at least one asymmetric unlined zone are selectively arranged in a series of intersecting and/or spaced spiral curves or patterns terminating at a common inner circle. A diameter of the ventilation holes can vary from a largest diameter near or adjacent outer edges of the anterior portion and/or the posterior portion gradually to a smallest diameter closer to the common inner circle. In some aspects, ventilation holes are positioned on an entire outer surface of the anterior and posterior portions, extending towards the outer edges and terminating at a solid portion running the entire perimeter of the orthosis. Ventilation holes are positioned at unlined zones on the inner surface of the anterior and posterior shells, terminating at the lined zone and/or the solid portion running the entire perimeter of the device. The ventilation holes advantageous allow for the child's or infant's head to be cooled and ventilated to allow for proper growth of the child's or infant's head.

In some aspects, in a connected state, an annular opening is formed between adjoining upper contoured edges of the anterior and posterior portions.

In some aspects, in a connected state, a slit is formed between adjoining lateral side edges of the anterior and posterior portions.

In some aspects, each of the anterior portion and the posterior portion include a pair of lateral edges extended between upper and lower contoured edges, and at least one connector positioned integrally on each lateral edge, each connector of the anterior portion configured to securely connect with a corresponding connector of the posterior portion.

In some aspects, the at least one connector of each lateral edge of the anterior portion is a tongue protruding orthogonally from the respective lateral edge.

In some aspects, the at least one connector of each lateral edge of the posterior portion is a receiving groove formed in the respective lateral edge and configured to prevent shear and securely receive a corresponding tongue of the anterior portion.

In some aspects, a securing mechanism configured to move between a connected state when the anterior and posterior portions are connected and a disconnected state when anterior and posterior portions are disconnected. The securing mechanism can include a perimetral ridge protruding from an outer surface of the anterior and posterior portions and surrounding an interior portion, and a clasp configured to securely engage a latch of the interior portion in the connected state and pivot away from the latch in the disconnected state.

In some aspects, a method is disclosed for producing a customized cranial deformation orthosis device for a skull of an infant. The method can include generating, by a three-dimensional scanner, a three-dimensional model of the skull of the infant based on a three-dimensional scan of the skull of the infant; analyzing, by a computing device, the three-dimensional model to determine one or more salient areas of the skull requiring reformation by the cranial deformation orthosis device; generating, by the computing device and based on the determined one or more salient areas, a corrected symmetrical shape model of the skull of the infant; and generating, by an additive fabricator and based on information of the corrected symmetrical shape model sent by the computing device, an anterior portion and a posterior portion of an outer shell of the customized cranial deformation orthosis device, each of the anterior portion and the posterior portion including an inner surface including at least one lined zone and at least one unlined zone configured to permit skull growth in a region of the skull aligned thereunder, the at least one unlined zone including a plurality of ventilation holes extended through the inner surface to an outer surface. The plurality of ventilation holes are open to allow air to easily reach the skull without any obstruction.

In some aspects, the method can include selectively positioned padding along the at least one lined zone of the posterior and/or anterior portions so as to prevent skull growth in one or more salient areas of the skull of the infant when the customized cranial deformation orthosis device is worn.

In some aspects, the step of analyzing, by the computing device, the three-dimensional model to determine one or more salient areas of the head requiring reformation by the cranial deformation orthosis device includes applying a machine learning system to the three-dimensional model to identify one or more salient areas and determine a deformation treatment protocol in connection with the corrected symmetrical shape model, the machine learning system having been generated by processing patient data and a plurality of historical training infant head models.

In some aspects, the step of generating, by the computing device and based on the determined one or more salient areas, the corrected symmetrical shape model of the skull of the infant includes overlaying one or more padding zones and at least one unlined zone of the anterior and posterior portions to reshape the skull of the infant. In this respect, the method can include detecting, by the computing device and the three-dimensional model, a cranial shape condition of the infant including at least one of symmetrical or asymmetrical brachycephaly, plagiocephaly, and symmetrical or asymmetrical scaphocephaly; and determining, by the computing device and based on the detected cranial shape condition, a padding arrangement to reshape the skull of the infant in a shape associated with the corrected symmetrical shape model.

In some aspects, the method can include the anterior and posterior portions of the outer shell are single monolithic units.

In some aspects, the additive fabricator of this disclosure can be at least one of powder bed fusion, binder jetting, material jetting, material extrusion, vat photopolymerization, laser-based stereolithography (SLA) systems, continuous liquid interface production (CLIP) systems, fused filament fabrication (FFF) systems, selective laser sintering (SLS) systems, and selective heat sintering (SHS).

In some aspects, the method can include selectively arranging the ventilation holes of the at least one unlined zone in a series of intersecting and/or spaced spiral curves or patterns terminating at a common inner circle, and wherein a diameter of the ventilation holes varies from a largest diameter near or adjacent outer edges of the anterior portion and/or the posterior portion gradually to a smallest diameter closer to the common inner circle.

In some aspects, the system for manufacturing a customized cranial deformation orthosis device includes at least one memory storing instructions and at least one processor configured to execute the instructions to perform operations, which can include generating a three-dimensional model of a skull of an infant based on a three-dimensional scan of the skull of the infant; analyzing the three-dimensional model to determine one or more salient areas of the skull requiring reformation by the cranial deformation orthosis device; generating, based on the determined one or more salient areas, a corrected symmetrical shape model of the skull of the infant; causing an additive fabricator to generate, based on information of the corrected symmetrical shape model, an anterior portion and a posterior portion of an outer shell of the customized cranial deformation orthosis device, each of the anterior portion and the posterior portion including an inner surface of at least one lined zone and at least one unlined zone including a plurality of ventilation holes extended through the inner surface to an outer surface.

To the accomplishment of the foregoing and related ends, certain illustrative aspects are described herein in connection with the following description and the appended drawings. These aspects are indicative, however, of but a few of the various ways in which the principles of the claimed subject matter may be employed and the claimed subject matter is intended to include all such aspects and their equivalents. Other advantages and novel features may become apparent from the following detailed description when considered in conjunction with the drawings.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation.

FIG. 1A shows a top plan view of an example head of an infant showing example bossed areas and flat areas requiring attention from an orthosis device.

FIG. 1B shows a side plan view of the example head of FIG. 1A showing bossed areas and flat areas requiring attention from an orthosis device.

FIG. 2 shows a perspective view of a cranial remodeling orthosis device according to certain aspects of this disclosure.

FIG. 3A shows a top plan schematic view of an example asymmetric head of an infant fit with an orthosis device according to certain aspects of this disclosure.

FIG. 3B shows a top plan schematic view of the example head of the infant of FIG. 3A after having been reshaped into a symmetric state fit with the orthosis device of FIG. 3A according to certain aspects of this disclosure.

FIG. 4A shows a perspective view of a posterior portion of an outer shell of an example cranial remodeling orthosis device according to certain aspects of this disclosure.

FIG. 4B shows a perspective view of an anterior portion of an outer shell of a cranial remodeling orthosis device according to certain aspects of this disclosure.

FIG. 5A shows a perspective view of a posterior portion of a cranial remodeling orthosis device with example padding selectively positioned therewith according to certain aspects of this disclosure.

FIG. 5B shows a perspective view of an anterior portion of a cranial remodeling orthosis device with example padding selectively positioned therewith according to certain aspects of this disclosure.

FIG. 6 shows a front plan cross-section view of the posterior portion of FIG. 4A according to certain aspects of this disclosure.

FIG. 7A shows a side perspective view of a cranial remodeling orthosis device with an example securing mechanism according to certain aspects of this disclosure.

FIG. 7B shows a side perspective view of a cranial remodeling orthosis device with another example securing mechanism according to certain aspects of this disclosure.

FIG. 8A shows a close-up of section 8A of FIG. 7B showing a close-up of the example securing mechanism in a connected state according to certain aspects of this disclosure.

FIG. 8B shows a close-up of section 8A of FIG. 7B showing a close-up of the example fastening mechanism in a disconnected state according to certain aspects of this disclosure.

FIG. 9 is a schematic drawing of a system for three-dimensional printing of a plurality of cranial remolding orthoses for remodeling a cranium in a corresponding plurality of stages.

FIG. 10 is a computer architecture diagram showing a computing system capable of implementing aspects of the present disclosure in accordance with one or more embodiments described herein.

FIG. 11A depicts a top plan view of example padding zones of a cranial remodeling orthosis device with padding positioned overlaid on an infant C with detected symmetrical brachycephaly.

FIG. 11B depicts a front plan view of the example padding zones of FIG. 11A overlaid on the infant.

FIG. 11C depicts a rear plan view of the example padding zones of FIG. 11A overlaid on the infant.

FIG. 11D depicts a side plan view of the example padding zones of FIG. 11A overlaid on the infant.

FIG. 12A depicts a top plan view of example padding zones of a cranial remodeling orthosis device with padding positioned overlaid on an infant C with detected combined brachycephaly and plagiocephaly.

FIG. 12B depicts a front plan view of the example padding zones of FIG. 12A overlaid on the infant.

FIG. 12C depicts a rear plan view of the example padding zones of FIG. 12A overlaid on the infant.

FIG. 12D depicts a side plan view of the example padding zones of FIG. 12A overlaid on the infant.

FIG. 13A depicts a top plan view of example padding zones of a cranial remodeling orthosis device with padding positioned overlaid on an infant C with detected symmetrical and asymmetrical scaphocephaly.

FIG. 13B depicts a front plan view of the example padding zones of FIG. 13A overlaid on the infant.

FIG. 13C depicts a rear plan view of the example padding zones of FIG. 13A overlaid on the infant.

FIG. 13D depicts a side plan view of the example padding zones of FIG. 13A overlaid on the infant.

### DETAILED DESCRIPTION

Although example embodiments of the disclosed technology are explained in detail herein, it is to be understood that other embodiments are contemplated. Accordingly, it is not intended that the disclosed technology be limited in its scope to the details of construction and arrangement of components set forth in the following description or illustrated in the drawings. The disclosed technology is capable of other embodiments and of being practiced or carried out in various ways.

It must also be noted that, as used in the specification and the appended claims, the singular forms “a,” “an” and “the” include plural referents unless the context clearly dictates otherwise. By “comprising” or “containing” or “including” it is meant that at least the named compound, element, particle, or method step is present in the composition or article or method, but does not exclude the presence of other compounds, materials, particles, method steps, even if the other such compounds, material, particles, method steps have the same function as what is named.

Specific embodiments are now described in detail with reference to the Figures, where similar reference numerals (e.g., 100 and 100′, 111 and 111′, etc.) indicate identical or similarly functional elements. In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the disclosed technology. Similarly, it is also to be understood that the mention of one or more components in a device or system does not preclude the presence of additional components or intervening components between those components expressly identified.

As discussed herein, a skull or head of a “subject” may be one of a young child such as an infant.

As discussed herein, “operator” may include, but is not limited to, a doctor, surgeon, nurse, physical therapist, or other healthcare professional, or any other suitable individual, or delivery instrumentation associated with the device of this disclosure.

As discussed herein, relative terms, such as “about,” “substantially,” or “approximately” are used to indicate a possible variation of ±10% in the stated value.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the disclosed technology. Similarly, it is also to be understood that the mention of one or more components in a device or system does not preclude the presence of additional components or intervening components between those components expressly identified.

As previously discussed, positional plagiocephaly is a disorder in which aspects of an infant's head becomes flattened (e.g., the back of the head, a left side of the head, a right side of the head, etc.). Typically, aspects of the infant's head becoming flattened is a consequence of the infant lying supine on their backs or otherwise positioned for extended periods of time so that the infant's head is resting against a flat surface in a manner that induces flattening (e.g., in a stroller, a car seat, a crib, a playpen, etc. playpens). Because the heads of infants are relatively soft and capable of being reshaped to allow for the brain growth that occurs in the first year of life, infants unfortunately are susceptible to being “molded” into a flat shape.

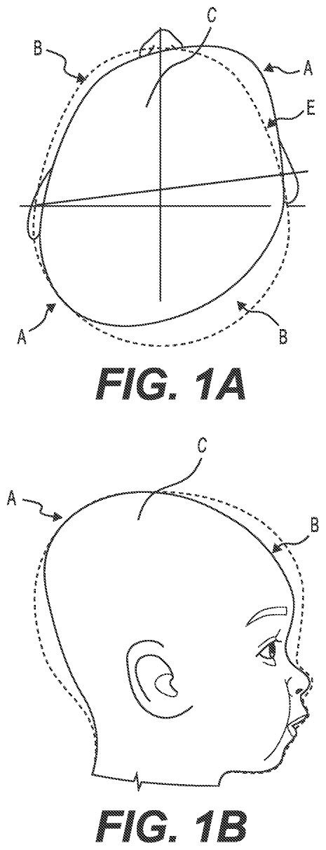

By way of example, FIG. 1A shows a top plan view and FIG. 1B shows a side plan view of an example head of an infant C showing example bossed areas A and flat areas B evidencing certain irregularities. As shown particularly with the denoted gaps between the exemplary symmetric overlay and flat areas B, the head of infant C shown in FIGS. 1A and 1B requires cranial remodeling from a cranial remodeling orthosis device to encourage infant C's head closer to the shape associated with the exemplary symmetric overlay.

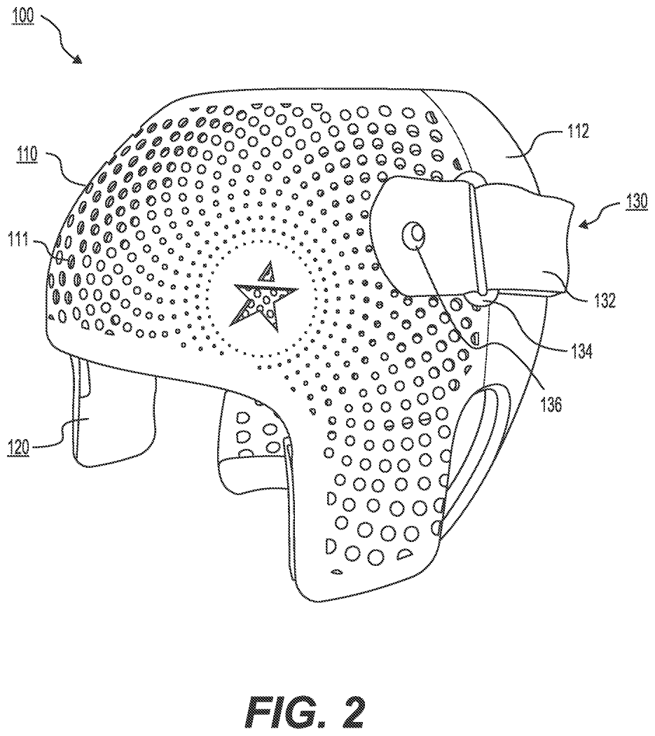

FIG. 2 shows a perspective view of a cranial remodeling orthosis device 100 according to certain aspects of this disclosure. Commonly referred to as a helmet, the device 100 of this disclosure is configured to resolve head shape deformities (e.g., correcting asymmetrical deformities) such as plagiocephaly, brachycephaly and scaphocephaly. In some aspects, device 100 is particularly effective in resolving head shape deformities in infants aged between approximately 3-18 months. The device 100 can be particularly optimal when used by younger infants (e.g., closer to 3 months) due to the decrease in growth rate as infants age. The device 100 is configured to hold or otherwise maintain total contact over selected areas where head growth is not desired while allowing for space over areas where head growth is desired. In turn, the device 100 is designed to capture the natural growth of a baby's head while inhibiting growth in the prominent areas (e.g., boss areas) and allowing for growth in one or more identified salient areas requiring reshaping by the device 100 (e.g., flat areas B of FIGS. 1A and 1B). In some aspects, the device 100 when worn by an infant is configured to provide a symmetrical space into which the head of the infant can grow. Even if the infant continues to rest their head on one side, the device 100 provides a controlled cushioning to prevent the infant's head from further flattening. In some aspects, the device 100 can include a rigid outer shell 110 (e.g., Nylon 12 (polymide 12)), with one or more padded zones 120 (e.g., padded cushions formed of foam) selectively positioned along an interior surface of a shell 110.

In some aspects, the one or more padded zones 120 are positioned or otherwise aligned with identified salient areas of the head of infant C (e.g., holding points or bossed areas of the head of infant C), allowing growth to occur in regions of the device 100 providing unlined void space. In so doing, otherwise excess material and weight of device 100 is reduced and breathability is promoted thereby reducing sweating while the device 100 is donned, unlike prior orthotic devices which provide complete padding and liner coverage on the infant. In some aspects, foam contemplated for use with the one or more padded zones 120 can include a closed-cell Ehtylene-Vinyl Acetate foam liner with uniform density and/or durometer. However, other foam materials are contemplated as well as use of varying density and/or durometer.

The shell 110 can be formed by two or more interconnected components. In some aspects, the two or more interconnected components of the shell 110 can be formed from an additively depositable thermoplastic material, including but not limited to one or more of acrylonitile butadine styrene, nylon, polyactic acid, polyvinyl alcohol, polycarbonate, polystyrene, polyetheylene terephthalate, and thermoplastic polyurethane. For example, the shell 110 may be formed by connecting a posterior portion 112 (e.g., a first half) with an anterior portion 111 (e.g., a second half). The portions 111 and 112 can be secured together with a securing mechanism 130. In some aspects, the mechanism 130 can include a strap 132 extended from one portion (e.g., the portion 112) to securely attach onto a corresponding receiver (e.g., a receiver positioned in this example on the portion 111). The receiver and strap 132 can each include hook and loop fastener surfaces, such as those provided by Velcro®. In other aspects, the mechanism 130 can include an anchor 136 fixedly attached (e.g., a rivet) to one portion of the shell 110 (e.g., the portion 112). The anchor 136 can include a belt loop 134 through which strap 132 can be inserted then wrapped backed so as to extend from the portion 112 and attach onto a receiver of the portion 111. Of course, aspects of the mechanism 130 as shown are merely exemplary and components of the mechanism 130 can be changed or even reversed (e.g., strap 132 can extend from the portion 111 to the portion 112, rather than from the portion 112 to the portion 111 as shown and described). Further, the portions 111 and 112 can be secured together by multiple mechanisms 130, rather than only one mechanism 130 as shown in FIG. 2, so as to securely engage the portions 111 and 112 to each other.

In some aspects, the portions 111 and 112 of shell 110 can each be selectively lined with padded zones 120. For example, the padded zones 120 can only be positioned in lined surfaces of the portions 111 and 112 so as to align over detected bossed areas of infant C (e.g., areas A of FIGS. 1A and 1B) while unpadded areas of the portions 111 and 112 can be positioned to align with or otherwise positioned over the flat areas (e.g., areas B of FIGS. 1A and 1B). In some aspects, once aspects of the shell 110 are created (e.g. created by process 900 discussed below), an interior surface of the shell 110 may include one or more areas which are unlined for the flat areas of the patient's head to grow into. In contrast, the padded zones 120 in the lined areas are configured to restrict undesirable growth in the bossed areas while allowing desired growth in the flat areas thus correcting the deformities. The padded zones 120 can also provide an opportunity for the operator to adjust an underlying area of an inner surface of the shell 110 to accommodate for normal growth. In some aspects, if the cranium of a respective infant C can grow into a zone unexpectedly (e.g., as a result of non-compliance by infant C), the operator can remove aspects of the padded zones 120 as needed (e.g., by selectively removing portions of the padded zones 120 or removing aspects of the padded zones 120 altogether).

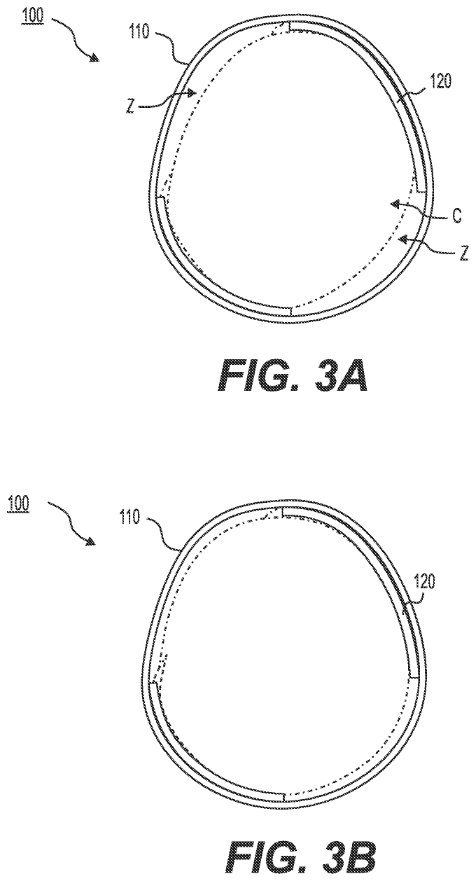

FIG. 3A shows a top plan cross-sectional schematic view of an example asymmetric head of an infant C donning an example device 100. As shown, padded zones 120 are positioned lined along an inner surface of shell 110 and in selective contact with areas (e.g., flat areas and/or bossed areas) of infant C's head. The shape and contours of the inner surface of the shell 110 are symmetric in nature and correspond to a determined symmetric shape for the specific infant C. Those portions of the padded zones 120 contacting infant C prevent skull growth. In contrast, those voided areas Z defined between infant C's head and the inner surface of shell 110 allow for skull growth therein. After the respective infant C has donned device 100 for a period of time (e.g., three months), FIG. 3B shows the example modified head of infant C where voided areas Z are now largely occupied by the corrected, symmetric head of infant C. Moreover, FIG. 3B also shows that the other areas of infant C's head have been prevented from growing by the padded zones 120.

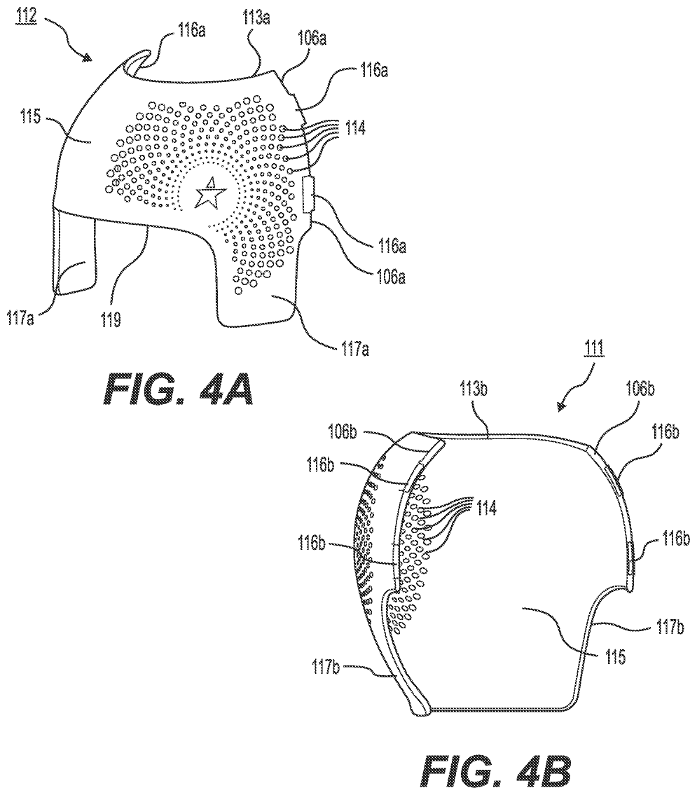

FIG. 4A shows a perspective view of an example posterior portion 112. In certain aspects, the portion 112 can include a lined portion 115 which can be contoured to surround aspects of a forehead of the head of infant C. In some aspects, some or all of an inner surface of the portion 115 of FIG. 4A can include padding of the padded zones 120. The portion 112 can also include a plurality of selectively positioned ventilation holes 114 positioned with unlined aspects of the portion 112. The unlined aspects or zone of the portion 112 can be generally asymmetric. For example, unlined aspects of the portion 112, including the holes 114, may only be positioned on one side of the portion 112. Unlined aspects of the portion 112 may also generally lack symmetry. In some aspects, the holes 114 can be arranged in a series of intersecting and/or spaced spiral curves or patterns terminating at a common inner circle, which are unlined.

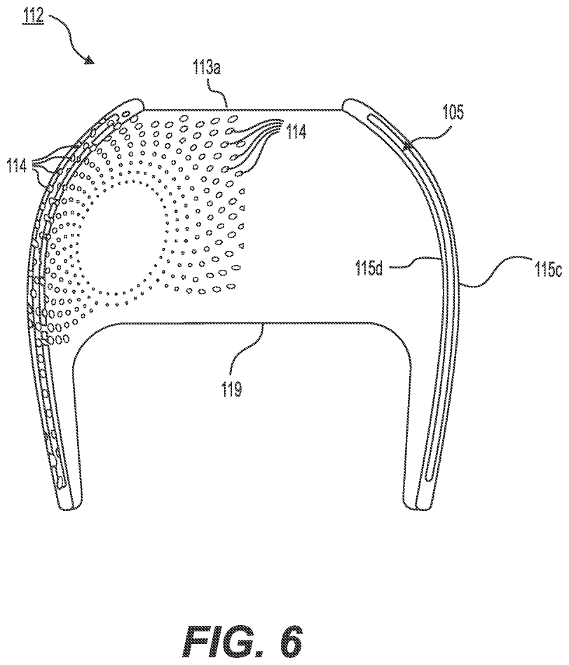

In some aspects, the holes 114 are incorporated into the portion 112 only in the unlined aspects and thus unpadded areas. In some aspects, the holes 114 do not penetrate and are not present through to the shell 110 in the lined areas to be padded (e.g., see FIG. 6). In some aspects, the holes 114 may all include the same diameter. In some aspects, the shape and/or the diameter of the holes 114 can vary from largest near or adjacent the outer edges gradually to smaller closer to the interior portion of the unlined aspects of the portions 112, 111. In some aspects, the diameter of the holes 114 can range in shape and diameter (e.g., outer larger shapes can be ellipse-like shapes (e.g., approximately 12 mm by 4 mm) to inner smallest circles having a diameter of approximately 0.35 mm). By only positioning holes 114 in unlined areas of shell 110, the inner surface of portions 111, 112 of the shell 110 can include a solid smooth surface to adhere the padding of the zones 120. Moreover, positioning the holes 114 in only unlined areas can serve as a guide for pad placement (e.g., when padding zones 120 are selected positioned and/or padding of the zones 120 are attached to the inner surface of the shell 110). The portion 112 may include a solid contoured edge 119 extended the entire perimeter of 111 and 112.

The portion 112 may include a plurality of connectors 116a integrally formed along an aft edge 106a. For example, a pair of connectors 116a may be positioned on a left side of the portion 112 and another pair of connectors 116a on an opposite, right side of the portion 112. In some aspects, each aft edge 106a having connectors 116a may extend longitudinally so as to be aligned on lower end adjacent an ear region of infant C towards a top of infant C's head. In some aspects, the portion 112 may include an upper solid contoured edge 113a extended between upper corners of the edges 106a and can form a semi-circular shape.

FIG. 4B shows a perspective view of an example anterior portion 111. In certain aspects and similar to the portion 112, the portion 111 can include a lined portion 115 which can be contoured so as to surround aspects of the aft portion of the head of infant C. In some aspects, some or all of the inner surface of the portion 115 of FIG. 4B can include padding of padded zones 120. Also similar to the portion 112, the portion 111 can also include a plurality of selectively positioned ventilation holes 114 positioned with unlined aspects of the portion 111, which can be similarly asymmetric in nature and appearance. In some aspects, the holes 114 are incorporated into the portion 111 only in the unlined and thus unpadded areas. The portion 111 may include a plurality of connectors 116b integrally formed along a forward edge 106b, whereby connectors 116b are configured to securely engage with corresponding respective connectors 116a of portion 112.

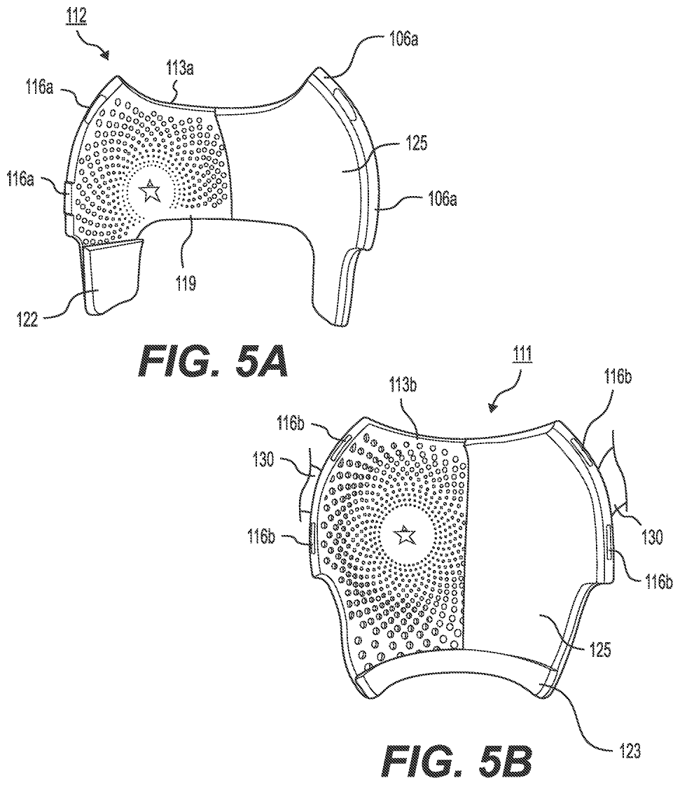

For example, connectors 116a, 116b can be tongue (e.g., a protrusion) and groove (e.g., a recess or other receiving surface) connectors configured to prevent shear while maintaining a smooth transition between the portions 111, 112. In FIG. 5A, example connectors 116a are shown protruding away from respective edges 106a while in FIG. 5B connectors 116b include recesses on respective edges 106b configured to form a friction fit with corresponding connectors 116a. In some aspects, once connected via respective connectors 116a, 116b, a continuous contoured outer surface can be formed between the portions 111 and 112. In some aspects, the portion 111 may include an upper contoured edge 113b extended between upper corners of edges 106b and can form a semi-circular shape. Once the portions 111 and 112 are connected together in a connected state, the edges 113a and 113b can form an annular opening (e.g., a substantially circular opening) in the upper surface of the shell 110. In some aspects, once donned by infant C, the opening formed between the edges 113a, 113b can expose an upper portion of the head of infant C thereby allowing for the head to continuing growing in the allotted space provided. As can be seen, once portions 111 and 112 are connected, the shell 110 is formed with a substantially lateral split therebetween.

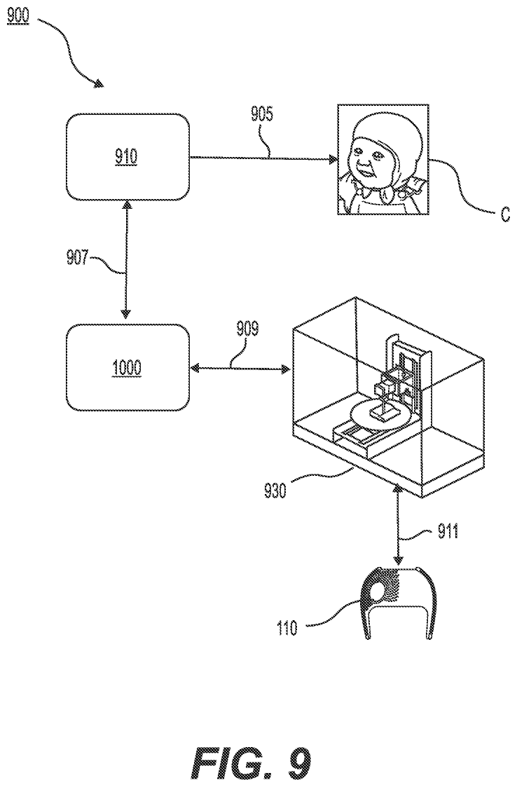

FIG. 5A shows a perspective view of portion 112 with example padding (e.g., padding 122, 125 of padding zone 120) selectively positioned along unlined aspects of the inner surface of the portion 112. By way of example only and not limitation, based on an exact three-dimensional scan of infant C's head and related analytics (e.g., see process 900 of FIG. 9), the padding 122 can be selectively positioned in a lower side portion adjacent edge 119 of lined aspects of the portion 112. Based also on the exact three-dimensional scan of infant C's head and related analytics, unlined aspects associated with the holes 114 can be selectively positioned. In some aspects, the padding 122 can be configured to redirect growth of the head of infant C in the corresponding identified salient areas of the head when the device 100 is worn. Similarly, the padding 125 can be positioned in the area opposite the unlined aspects of the inner surface. In some aspects, the padding 125 can extend between unlined aspects of the holes 114, the edge 113a, and the edge 119. Similar to the padding 122, the padding 125 can be configured to apply a direct holding point to infant C so as to prevent further growth of the head of infant C in the corresponding head region when the device 100 is worn.

FIG. 5B shows a perspective view of the portion 111 with example padding (e.g., padding 123, 125 of padding zone 120) selectively positioned along unlined aspects of the inner surface of the portion 111. In some aspects, the padding 123 can be positioned along a lower edge of the portion 111 between opposing lateral edges and underneath unlined aspects of the holes 114. Both the paddings 123 and 125 of FIG. 5B can apply a direct contact holding point to infant C when the device 100 is worn so as to prevent further growth of the head of infant C in the corresponding head region.

FIG. 6 shows a front plan cross-section view of the portion 112 of FIG. 4A taken along a mid-section according to certain aspects of this disclosure. As shown, the portion 112 can be hollow with a void 105 formed between the inner surface 115d and the outer surface 115c of the lined portion 115. In some aspects, a thickness of the walls of the inner surface 115d and the outer surface 115c can be approximately 1.25 mm and the void 105 can be approximately 2 mm so that a total thickness of the portion 112 (and the portion 111) can be 4.5 mm. A similar void can exist between inner and outer surfaces of unlined portions, including with the holes 114. The void 105 is particularly advantageous as it reduces the weight of the device 100 and thus making it more comfortable for infant C wearing the device 100.

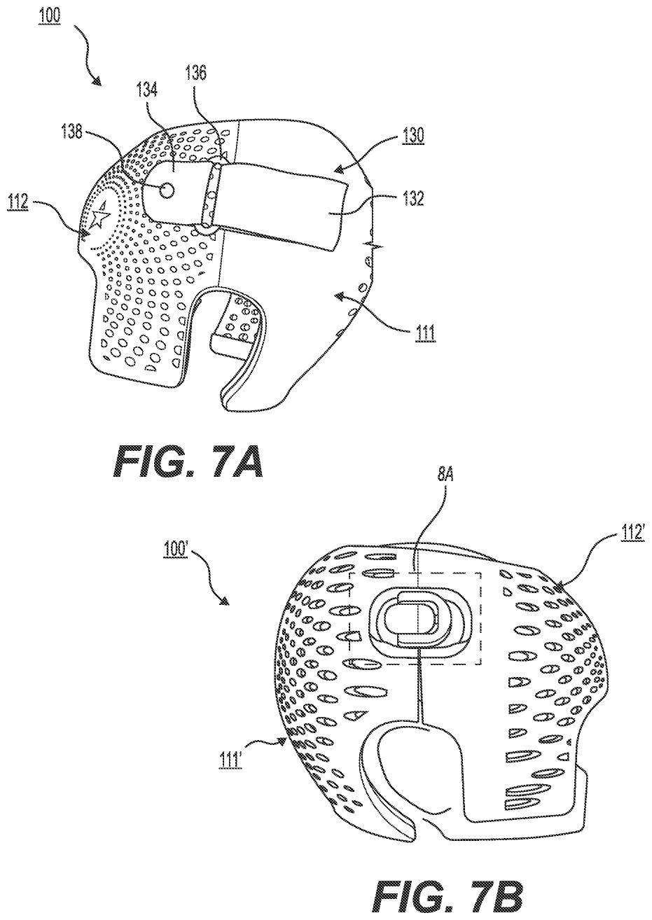

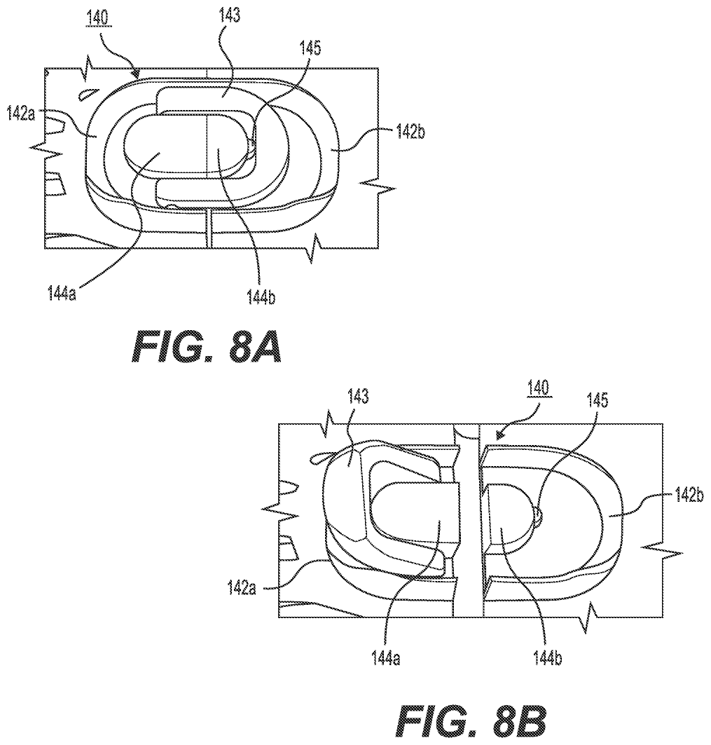

FIG. 7A shows a side perspective view of device 100 with previously described securing mechanism 130 in a secured state so that the portions 111 and 112 are secured together. Once secured together in the connected state, the portions 111 and 112 provided the described smooth transition therebetween. FIG. 7B shows a side perspective view of another example the device 100′ with another example securing mechanism 140 shown in section 8A, instead of and/or in addition to the previously discussed securing mechanism 130. Turning to FIG. 8A, a close-up is shown of section 8A of FIG. 7B showing a close-up of the example securing mechanism 140 in a connected state. The mechanism 140 can be an integrated clasping mechanism.

The mechanism 140 can be integrally formed with the portions 111′, 112′ and positioned on adjoining edges of the lateral slit that runs between the portions 111′, 112′ when connected to the other. The mechanism 140 can include a first perimetral ridge 142a of portion 111′ and a second perimetral ridge 142b of portion 112′. Each of ridges 142a, 142b can extend radially outward from respective outer surfaces of the portions 111′, 112′. Each of the ridges 142a, 142b can include a partial elliptical or semi-circular shape so that when the portions 111′, 112′ are connected together a corresponding outer shape is formed between the ridges 142a, 142b (e.g., an outer ellipse, an outer circle, or any other shape as needed or desired). The mechanism 140 can also include a first interior portion 144a of the portion 111′ and a second interior portion 144b of the portion 112′. Each of the portions 144a, 144b can also extend outward radially and be positioned within and/or inset from respective ridges 142a, 142b. Each of portions 144a, 144b can include a partial elliptical or semi-circular shape so that when portions 111′, 112′ are connected together, a corresponding outer shape is formed between the portions 144a, 144b (e.g., an outer ellipse, an outer circle, or any other shape as needed or desired). In some aspects, the portion 144b can also include a latch 145 on edge opposite portion 144a. A clasp 143 may be pivotably connected to one or more edges of the portion 144a. In the connected state of FIG. 8A, the clasp 143 is shown securely retained underneath aspects of the latch 145 so that the smooth transition is formed between the portions 111′, 112′. In contrast, in the disconnected state of FIG. 8B, the clasp 143 has pivoted about a central pivot of the portion 144a (e.g., about a hinge therein) so that the clasp 143 is released from the latch 145. Once in the disconnected state, it can be seen that a space is provided between the portions 111′, 112′ so that the smooth transition is no longer present therebetween. Of course, the example of FIGS. 7B to 8B is merely one embodiment and aspects can be reversed or reordered (e.g., the latch 145 can be on the portion 144a and the clasp 143 can be pivotably connected to the portion 144b).

FIG. 9 depicts an example process 900 of manufacturing a customized orthosis according to certain aspects of this disclosure. In a step 905, an infant C may have their head scanned by a three-dimensional scanner 910 to generate an exact digital three-dimensional model of infant C's head. In a step 907, once generated, the three-dimensional model is electronically sent to a computing device of a manufacturing device 1000, such as an additive fabricator 930 otherwise known as “three-dimensional printing” system.

In step 909, device 1000 can analyze (e.g., semi- and/or completely automatically) the exact digital three-dimensional model to determine one or more salient areas of infant C's head require reformation by the orthosis. In some aspects, computing methods used to determine the one or more salient areas of infant C's head and may include, but are not limited to, statistical analysis, autonomous or machine learning, and AI. AI may include, but is not limited to, deep learning, neural networks, classifications, clustering, and regression algorithms. By using such computing methods, head diagnostic accuracy is substantially improved as is reliability and efficiency. In some aspects, a computing system operating one or more of the foregoing computing methods can include a trained machine learning algorithm that takes, as input, data of the infant's head as well as historical training infant head models, and historical patient data, and determines one or more salient areas thereof (e.g., bossed area, flattened area, etc.) requiring reshaping by the deformation orthosis device.

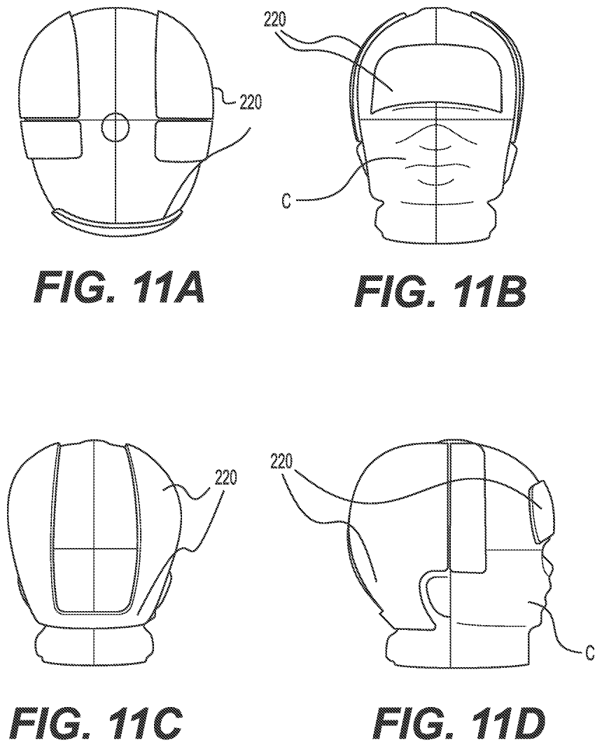

Many methods may be used to learn which areas are salient, including but not limited to: (1) weak supervision: training a machine learning system (e.g., multi-layer perceptron (MLP), convolutional neural network (CNN), graph neural network, support vector machine (SVM), random forest, etc.) using multiple instance learning (MIL) using weak labeling of the digital image or a collection of images; the label may correspond to the presence or absence of a salient areas; (2) bounding box or polygon-based supervision: training a machine learning system (e.g., region-based CNN (R-CNN), Faster R-CNN, Selective Search) using bounding boxes or polygons that specify the sub-regions of the digital image that are salient for the detection of the presence or absence of the biomarker; (3) pixel-level labeling (e.g., a semantic or instance segmentation): training a machine learning system (e.g., Mask R-CNN, U-Net, Fully Convolutional Neural Network) using a pixel-level labeling, where individual pixels are identified as being salient; and/or (4) using a corresponding, but different digital image that identifies salient area. Based on determining the one or more salient areas, device 1000 can generate a corrected symmetrical shape model of the head of the infant C. For example, the model can include information related to aspects of the outer shell of an associate orthosis (e.g., size and shape of posterior portions, anterior portions, lined zones, unlined zones, and locations of padding materials required for reformation of the head of infant C, etc.). In some aspects, the machine learning system can be trained to detect, based on the three-dimensional model, a cranial shape condition of the infant (e.g., at least one of symmetrical brachycephaly, brachycephaly and plagiocephaly, and symmetrical and asymmetrical scaphocephaly). In some aspects, the machine learning system can be trained to also determine, determining, based on the detected cranial shape condition, a padding arrangement (e.g., see those example, non-limiting arrangements in FIGS. 11-13) to reshape the head of the infant in a shape associated with the corrected symmetrical shape model. Based on the generated model, padding material can be added to the determined one or more salient areas so as to reform the flat areas infant C's head rather than removing material from the bossed areas infant C's head. In step 909, device 1000 can then generate an updated three-dimensional model of the actual orthosis over an underlying corrected shape.

In step 911, based on the updated corrected symmetrical shape model, one or more shell models are created by fabricator 930 to create the anterior 111 and posterior 112 portions of shell 110. In some aspects, anterior 111 and posterior 112 portions of shell 110 are single monolithic units or otherwise integrally formed.

In some aspects of process 900, once the orthosis shell 110 is fabricated, it can be packaged in a kit along with a padding kit that corresponds with the size of the orthosis shell 110 as well as the correction desired in infant C's head. In some aspects, the packaged kit which can include orthosis shell 110 and corresponding padding can be shipped to an operator (e.g., the clinician) for fitting the device to the respective head of infant C.

In some aspects of process 900, the operator can selectively position the padding material (e.g., foam pads) included in the padding kit only in the determined one or more areas that corresponds to the bossed areas of the child's head. Each pad has a pressure sensitive adhesive on the underlying side for affixing to the inside of orthosis shell 110. While portions of orthosis shell 110 can be perforated for ventilation (e.g., via holes 114), the remaining portions of orthosis shell 110 can be smooth relatively along the inner surface (e.g., surface 115d) to accommodate optimal adhesion to padding material. In some aspects, as infant C grows, some or all the padding material previously added in the determined one or more areas may be removed to accommodate changes in infant C's head shape (e.g., by grinding away material, removing the padding altogether, etc.).

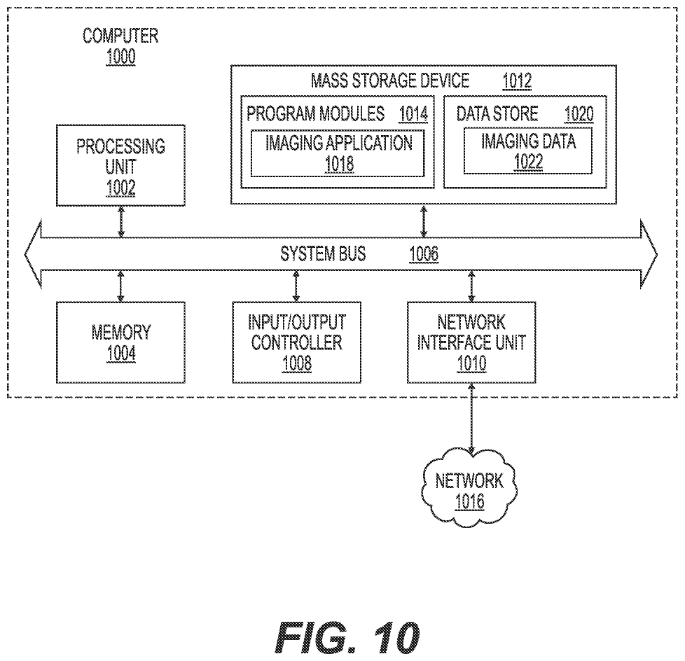

FIG. 10 is a computer architecture diagram showing a general computing system capable of implementing aspects of the present disclosure in accordance with one or more embodiments described herein. A computer 1000 of the aforementioned manufacturing example process 900 shown in FIG. 9 may be configured to perform one or more functions associated with embodiments of this disclosure. For example, the computer 1000 may be configured to perform operations in order to process three-dimensional scanned information related to a specific child's C head and coordinate fabrication of an orthosis device (e.g., device 100, 200, 300, 400, etc.) specific to an exact scan of infant C's head. It should be appreciated that the computer 1000 may be implemented within a single computing device or a computing system formed with multiple connected computing devices. The computer 1000 may be configured to perform various distributed computing tasks, in which processing and/or storage resources may be distributed among the multiple devices. The data acquisition and display computer 1050 and/or operator console 1010 of the system shown in FIG. 10 may include one or more systems and components of the computer 1000.

As shown, the computer 1000 includes a processing unit 1002 (“CPU”), a system memory 1004, and a system bus 1006 that couples the memory 1004 to the CPU 1002. The computer 1000 further includes a mass storage device 1012 for storing program modules 1014. The program modules 1014 may be operable to analyze and/or modify current settings of the applicator, as well as individualize aspects of an orthosis of an infant C, including aspects of the outer shell, padding material, and respective dimensions and locations of each. The program modules 1014 may include an imaging application 1018 for performing data acquisition and/or processing functions as described herein, for example to acquire and/or process image data corresponding to the three-dimensional scan of infant C's head. The computer 1000 can include a data store 1020 for storing data that may include imaging-related data 1022 such as acquired data from the implementation of magnetic resonance imaging in accordance with various embodiments of the present disclosure.

The mass storage device 1012 is connected to the CPU 1002 through a mass storage controller (not shown) connected to the bus 1006. The mass storage device 1012 and its associated computer-storage media provide non-volatile storage for the computer 1000. Although the description of computer-storage media contained herein refers to a mass storage device, such as a hard disk or CD-ROM drive, it should be appreciated by those skilled in the art that computer-storage media can be any available computer storage media that can be accessed by the computer 1000.

By way of example and not limitation, computer storage media (also referred to herein as “computer-readable storage medium” or “computer-readable storage media”) may include volatile and non-volatile, removable and non-removable media implemented in any method or technology for storage of information such as computer-storage instructions, data structures, program modules, or other data. For example, computer storage media includes, but is not limited to, RAM, ROM, EPROM, EEPROM, flash memory or other solid state memory technology, CD-ROM, digital versatile disks (“DVD”), HD-DVD, BLU-RAY, or other optical storage, magnetic cassettes, magnetic tape, magnetic disk storage or other magnetic storage devices, or any other medium which can be used to store the desired information and which can be accessed by the computer 1000. “Computer storage media”, “computer-readable storage medium” or “computer-readable storage media” as described herein do not include transitory signals.

According to various embodiments, the computer 1000 may operate in a networked environment using connections to other local or remote computers through a network 1016 via a network interface unit 1010 connected to the bus 1006. The network interface unit 1010 may facilitate connection of the computing device inputs and outputs to one or more suitable networks and/or connections such as a local area network (LAN), a wide area network (WAN), the Internet, a cellular network, a radio frequency (RF) network, a Bluetooth-enabled network, a Wi-Fi enabled network, a satellite-based network, or other wired and/or wireless networks for communication with external devices and/or systems.

The computer 1000 may also include an input/output controller 1008 for receiving and processing input from any of a number of input devices. Input devices may include one or more of keyboards, mice, stylus, touchscreens, microphones, audio capturing devices, and image/video capturing devices. An end user may utilize the input devices to interact with a user interface, for example a graphical user interface, for managing various functions performed by the computer 1000. The bus 1006 may enable the processing unit 1002 to read code and/or data to/from the mass storage device 1012 or other computer-storage media.

The computer-storage media may represent apparatus in the form of storage elements that are implemented using any suitable technology, including but not limited to semiconductors, magnetic materials, optics, or the like. The computer-storage media may represent memory components, whether characterized as RAM, ROM, flash, or other types of technology. The computer storage media may also represent secondary storage, whether implemented as hard drives or otherwise. Hard drive implementations may be characterized as solid state or may include rotating media storing magnetically-encoded information. The program modules 1014, which include the imaging application 1018, may include instructions that, when loaded into the processing unit 1002 and executed, cause the computer 1000 to provide functions associated with one or more embodiments illustrated in the figures of this disclosure. The program modules 1014 may also provide various tools or techniques by which the computer 1000 may participate within the overall systems or operating environments using the components, flows, and data structures discussed throughout this description.

In general, the program modules 1014 may, when loaded into the processing unit 1002 and executed, transform the processing unit 1002 and the overall computer 1000 from a general-purpose computing system into a special-purpose computing system. The processing unit 1002 may be constructed from any number of transistors or other discrete circuit elements, which may individually or collectively assume any number of states. More specifically, the processing unit 1002 may operate as a finite-state machine, in response to executable instructions contained within the program modules 1014. These computer-executable instructions may transform the processing unit 1002 by specifying how the processing unit 1002 transitions between states, thereby transforming the transistors or other discrete hardware elements constituting the processing unit 1002.

Encoding the program modules 1014 may also transform the physical structure of the computer-storage media. The specific transformation of physical structure may depend on various factors, in different implementations of this description. Examples of such factors may include but are not limited to the technology used to implement the computer-storage media, whether the computer storage media are characterized as primary or secondary storage, and the like. For example, if the computer storage media are implemented as semiconductor-based memory, the program modules 1014 may transform the physical state of the semiconductor memory, when the software is encoded therein. For example, the program modules 1014 may transform the state of transistors, capacitors, or other discrete circuit elements constituting the semiconductor memory.

As another example, the computer storage media may be implemented using magnetic or optical technology. In such implementations, the program modules 1014 may transform the physical state of magnetic or optical media, when the software is encoded therein. These transformations may include altering the magnetic characteristics of particular locations within given magnetic media. These transformations may also include altering the physical features or characteristics of particular locations within given optical media, to change the optical characteristics of those locations. Other transformations of physical media are possible without departing from the scope of the present description, with the foregoing examples provided only to facilitate this discussion.

FIGS. 11A to 13D show examples different cranial deformities with corresponding pad kits and selective placement. Specifically, FIG. 11A to FIG. 11D depicts views of example padding zones 220 of a cranial remodeling orthosis device 200 with padding positioned overlaid on an infant C with detected symmetrical brachycephaly. In some aspects, aspects of process 900 have been performed whereby it has been determined, based on the scan of infant C, that infant C has symmetrical brachycephaly and aspects of device 200 have been thereby generated. Padding zones 220 of device 200 are selectively positioned in FIG. 11A to FIG. 11D in an optimized arrangement to corresponding regions of infant C's head to prevent further growth in areas associated therewith. For example, the padding scheme of zones 220 correspond with preventing further growth in the forehead region, lower rear neck region, and rear lateral region of infant C's head.

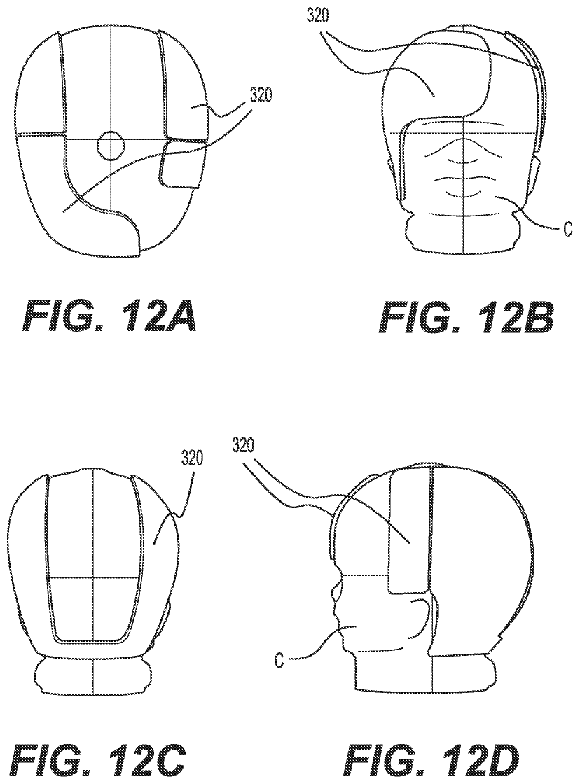

FIG. 12A to FIG. 12D depicts views of example padding zones 320 of a cranial remodeling orthosis device 300 with padding positioned overlaid on an infant C with detected asymmetrical brachycephaly and/or combined brachycephaly and plagiocephaly. In some aspects, aspects of process 900 have been performed whereby it has been determined, based on the scan of infant C, that infant C has detected asymmetrical brachycephaly and/or combined brachycephaly and plagiocephaly and aspects of device 300 have been thereby generated. Padding zones 320 of device 300 are selectively positioned in FIG. 12A to FIG. 12D in an optimized arrangement to corresponding regions of infant C's head to prevent further growth in areas associated therewith. For example, the padding scheme of zones 320 correspond with preventing further growth in the forehead region, lower rear neck region, and rear lateral region of infant C's head.

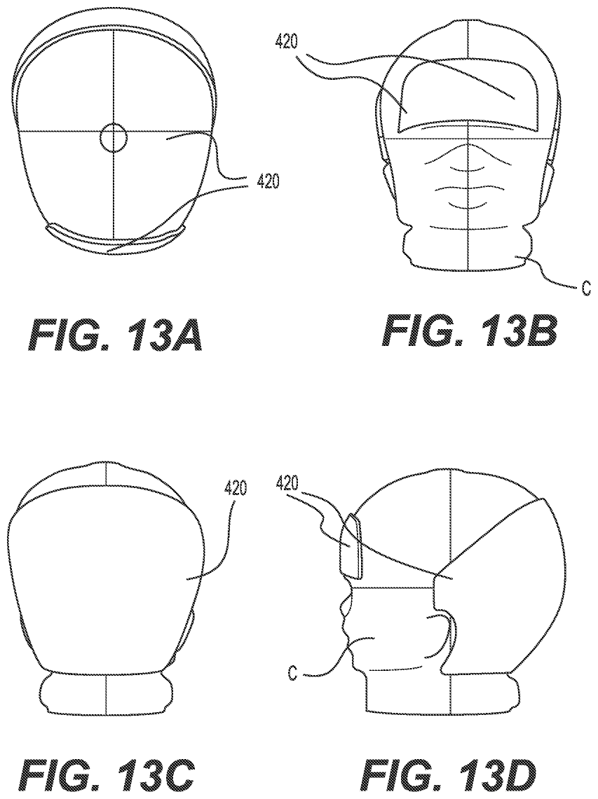

FIG. 13A to FIG. 13D depicts views of example padding zones 420 of a cranial remodeling orthosis device 400 with padding positioned overlaid on an infant C with detected symmetrical and asymmetrical scaphocephaly. In some aspects, aspects of process 900 have been performed whereby it has been determined, based on the scan of infant C, that infant C has detected symmetrical and asymmetrical scaphocephaly and aspects of device 400 have been thereby generated. The padding zones 420 of device 400 are selectively positioned in FIG. 12A to FIG. 12D in an optimized arrangement to corresponding regions of infant C's head to prevent further growth in areas associated therewith. For example, the padding scheme of zones 420 correspond with preventing further growth in the forehead region, lower rear neck region, and rear lateral region of infant C's head.

The specific configurations, choice of materials and the size and shape of various elements can be varied according to particular design specifications or constraints requiring a system or method constructed according to the principles of the disclosed technology. Such changes are intended to be embraced within the scope of the disclosed technology. The presently disclosed embodiments, therefore, are considered in all respects to be illustrative and not restrictive. It will therefore be apparent from the foregoing that while particular forms of the disclosure have been illustrated and described, various modifications can be made without departing from the spirit and scope of the disclosure and all changes that come within the meaning and range of equivalents thereof are intended to be embraced therein.

What is claimed is:

1. A cranial deformation orthosis device for a skull of an infant, comprising:
a rigid outer shell having an anterior portion detachably connected to a posterior portion, the anterior portion being shaped and configured to be donned on a forward portion of the skull of the infant and the posterior portion being shaped and configured to be donned on an aft portion of the skull of the infant; and
each of the anterior portion and the posterior portion has an inner surface with at least one lined zone having selectively positioned padding to prevent skull growth in a region of the skull aligned thereunder and at least one unlined zone configured to permit skull growth in a region of the skull aligned thereunder, the at least one unlined zone having a plurality of ventilation holes extended through the inner surface to an outer surface.

2. The device of claim 1, wherein each of the anterior portion and the posterior portion are hollow with a void formed between inner and outer surfaces, and wherein the at least one unlined zone is asymmetric.

3. The device of claim 1, wherein a size and shape of the at least one lined zone is assigned based on data of an exact three-dimensional scan of the skull of the infant, wherein the selectively positioned padding is configured to apply a direct holding point to the skull of the infant so as to prevent further growth of the skull in a corresponding skull region when device is worn.

4. The device of claim 1, wherein a size and a shape of the at least one unlined zone is assigned based on data of an exact three-dimensional scan of the skull of the infant.

5. The device of claim 4, wherein the ventilation holes of the at least one unlined zone are selectively arranged in a series of intersecting and/or spaced spiral curves or patterns terminating at a common inner circle, and wherein a diameter of the ventilation holes varies from a largest diameter near or adjacent outer edges of the anterior portion and/or the posterior portion gradually to a smallest diameter closer to the common inner circle.

6. The device of claim 1, wherein in a connected state, an annular opening is formed between adjoining upper contoured edges of the anterior and posterior portions.

7. The device of claim 1, wherein in a connected state, a slit is formed between adjoining lateral side edges of the anterior and posterior portions.

8. The device of claim 1, wherein each of the anterior portion and the posterior portion comprise:
a pair of lateral edges extended between upper and lower contoured edges; and
at least one connector positioned integrally on each lateral edge, each connector of the anterior portion configured to securely connect with a corresponding connector of the posterior portion.

9. The device of claim 8, wherein the at least one connector of each lateral edge of the anterior portion is a tongue protruding orthogonally from the respective lateral edge.

10. The device of claim 9, wherein the at least one connector of each lateral edge of the posterior portion is a receiving groove formed in the respective lateral edge and configured to prevent shear and securely receive a corresponding tongue of the anterior portion.

11. The device of claim 1, further comprising:
a securing mechanism configured to move between a connected state when anterior and posterior portions are connected and a disconnected state when anterior and posterior portions are disconnected, the securing mechanism comprising a perimetral ridge protruding from an outer surface of the anterior and posterior portions and surrounding an interior portion, and a clasp configured to securely engage a latch of the interior portion in the connected state and pivot away from the latch in the disconnected state.

12. A method for producing a customized cranial deformation orthosis device for a skull of an infant, comprising:
generating, by a three-dimensional scanner, a three-dimensional model of the skull of the infant based on a three-dimensional scan of the skull of the infant;
analyzing, by a computing device, the three-dimensional model to determine one or more salient areas of the skull requiring reformation by the cranial deformation orthosis device;
generating, by the computing device and based on the determined one or more salient areas, a corrected symmetrical shape model of the skull of the infant; and
generating, by an additive fabricator and based on information of the corrected symmetrical shape model sent by the computing device, an anterior portion and a posterior portion of an outer shell of the customized cranial deformation orthosis device, each of the anterior portion and the posterior portion has an inner surface having at least one lined zone and at least one unlined zone configured to permit skull growth in a region of the skull aligned thereunder, the at least one unlined zone including a plurality of ventilation holes extended through the inner surface to an outer surface.

13. The method of claim 12, further comprising:
selectively positioned padding along the at least one lined zone of the posterior and/or anterior portions so as to prevent skull growth in one or more of the one or more salient areas of the skull of the infant when the customized cranial deformation orthosis device is worn.

14. The method of claim 12, wherein the step of analyzing, by the computing device, the three-dimensional model to determine one or more salient areas of the skull requiring reformation by the cranial deformation orthosis device comprises applying a machine learning system to the three-dimensional model to identify one or more salient areas and determine a deformation treatment protocol in connection with the corrected symmetrical shape model, the machine learning system having been generated by processing patient data and a plurality of historical training infant skull models.

15. The method of claim 12, wherein the step of generating, by the computing device and based on the determined one or more salient areas, the corrected symmetrical shape model of the skull of the infant comprises overlaying one or more padding zones in the at least one unlined zone of the anterior and posterior portions to reshape the skull of the infant, the method further comprising:
detecting, by the computing device and the three-dimensional model, a cranial shape condition of the infant comprising at least one of symmetrical brachycephaly, brachycephaly and plagiocephaly, and symmetrical and asymmetrical scaphocephaly; and
determining, by the computing device and based on the detected cranial shape condition, a padding arrangement to reshape the head of the infant in a shape associated with the corrected symmetrical shape model.

16. The method of claim 12, wherein the anterior and posterior portions of the outer shell are single monolithic units.

17. The method of claim 12, wherein the additive fabricator includes at least one of laser-based stereolithography (SLA) systems, continuous liquid interface production (CLIP) systems, fused filament fabrication (FFF) systems, selective laser sintering (SLS) systems or selective heat sintering (SHS).

18. The method of claim 12, further comprising:
selectively arranging the ventilation holes of the asymmetric unlined zone in a series of intersecting and/or spaced spiral curves or patterns terminating at a common inner circle, and wherein a diameter of the ventilation holes varies from a largest diameter near or adjacent outer edges of the anterior portion and/or the posterior portion gradually to a smallest diameter closer to the common inner circle.

19. A system for manufacturing a customized cranial deformation orthosis device, the system comprising:
at least one memory storing instructions; and
at least one processor configured to execute the instructions to perform operations comprising:
generating a three-dimensional model of a head of an infant based on a three-dimensional scan of the head of the infant;
analyzing the three-dimensional model to determine one or more salient areas of the head requiring reformation by the cranial deformation orthosis device;
generating, based on the determined one or more salient areas, a corrected symmetrical shape model of the head of the infant; and
causing an additive fabricator to generate, based on information of the corrected symmetrical shape model, an anterior portion and a posterior portion of an outer shell of the customized cranial deformation orthosis device, each of the anterior portion and the posterior portion has an inner surface having at least one lined zone and at least one asymmetric unlined zone comprising a plurality of ventilation holes extended through the inner surface to an outer surface.

20. The system of claim 19, wherein the step of generating, based on the determined one or more salient areas, the corrected symmetrical shape model of the head of the infant comprises overlaying one or more padding zones in the at least one asymmetric unlined zone of the anterior and posterior portions to reshape the head of the infant.
